(19) United States
(12) Patent Application Publication
(10) Pub. No.: US 2013/0236359 A1
(43) Pub. Date: Sep. 12, 2013

(54) METHODS FOR STERILIZATION IN A VACUUM SYSTEM

(71) Applicant: Christopher Wayne Burger, Middletown, NY (US)

(72) Inventor: Christopher Wayne Burger, Middletown, NY (US)

(73) Assignee: SP INDUSTRIES, INC., Warminster, PA (US)

(21) Appl. No.: 13/872,709

(22) Filed: Apr. 29, 2013

Related U.S. Application Data
(62) Division of application No. 13/178,543, filed on Jul. 8, 2011.
(60) Provisional application No. 61/363,019, filed on Jul. 9, 2010.

(51) Int. Cl. A61L 2/20

(52) U.S. Cl. CPC A61L 2/20
USPC 422/33

(57) ABSTRACT

A method of sterilizing a vacuum system, comprising: creating a vacuum in a vacuum chamber such that a pressure differential is formed between the vacuum chamber and a liquid sterilant supply, and wherein the liquid sterilant supply has a higher pressure than the vacuum chamber; vaporizing liquid sterilant; and sterilizing the vacuum chamber with the vaporized sterilant.

### CROSS REFERENCE TO RELATED APPLICATIONS

This application is a divisional of U.S. patent application Ser. No. 13/178,543, filed on Jul. 8, 2011, which claims priority to U.S. Patent Application Ser. No. 61/363,019, filed Jul. 9, 2010, the contents of which are herein incorporated by reference in its entirety.

### BACKGROUND

This disclosure relates to vacuum systems and particularly to vacuum systems requiring sterilization and methods for using the same.

Vacuum systems are used in many industries for many processes. Where the system is utilized in food, biological or pharmaceutical applications sterilization of the system is often required.

For example, lyophilization or freeze drying is a process in which water and/or other solvents are removed from a product. After it is frozen, a vacuum is applied, allowing the ice to change directly from solid to vapor without passing through a liquid phase. The process consists of three separate, unique, and interdependent processes; freezing, primary drying (sublimation), and secondary drying (desorption). Lyophilization is generally used as a means to preserve materials making them more stable and easy to reconstitute. Once the process has been completed, the system is sterilized.

One sterilization technique involves the use of high temperature, high pressure steam. Typically this requires supplying steam (at about 125° C. and 30 pounds per square inch (psi), and introducing the steam into the lyophilizer chambers and all other internally wetted surfaces for a defined period of time. This is performed after the lyophilization cycle is completed. The lyophilized vials are stoppered, the chamber is then returned to atmospheric pressure, and the stoppered vials are removed. The system is then ready for the sterilization cycle. Problems with this method include that the lyophilizer must be built to withstand temperature and pressure extremes, the attendant additional machine costs and leadtimes, wear and tear on equipment (thermal and pressure cycling), and extended cycle times (heat up and cool down), which negatively impact productivity, and the requirements of facilities infrastructures and utility costs to generate an ample supply of clean steam.

Another sterilization technique involves the gas ethylene oxide (‘ETO’), which can be injected into a lyophilizer at subatmospheric pressure to accomplish terminal sterilization. Problems with this method include the fact that ETO is a toxic, flammable substance. Hence, regulations, health safety concerns, and market preferences are increasingly favoring steam sterilization.

Yet another sterilization technique involves the chemical hydrogen peroxide (‘H₂O₂’) in a water solution, which is also toxic to most biologics. Current H₂O₂ sterilization processes involve the use of a purpose-built device to vaporize (with heat) liquid H₂O₂. The vaporized H₂O₂ is injected into the lyophilizer at or slightly below atmospheric pressure and then circulated in a closed loop between the lyophilizer and the H₂O₂ vaporizer for a period of time, at specified temperatures, and at sufficient concentrations to kill the targeted biologics. Issues with this method include it requiring the use of an expensive H₂O₂ generator, care must be taken to not pressurize the lyophilizer, which is designed and rated only for vacuum conditions and difficulties in achieving sufficient coverage of the sterilant onto all internally wetted surfaces.

Hence there exists a continuing need in the art for an efficient, effective technique and system for sterilizing vacuum systems (e.g., lyophilizer systems) that is not plagued by the issues of over pressurization.

### BRIEF DESCRIPTION

The above-described drawbacks are alleviated by the vacuum systems and method for sterilization disclosed herein.

In one embodiment, a vacuum system comprises: a vacuum chamber; a vacuum source in operable communication with the vacuum chamber; a liquid sterilant supply; and a metering device. The sterilant supply is in operable communication with the vacuum chamber via the metering device. The metering device is sized such that liquid sterilant can be introduced into the vacuum chamber as sterilant vapor.

In one embodiment, a method of sterilizing a vacuum system comprises: creating a vacuum in a vacuum chamber such that a pressure differential is formed between the vacuum chamber and a liquid sterilant supply, and wherein the liquid sterilant supply has a higher pressure than the vacuum chamber; vaporizing liquid sterilant; and sterilizing the vacuum chamber with the vaporized sterilant.

In one embodiment, a method of lyophilization comprises: loading a material into a vacuum chamber having a chamber pressure, the material having a material eutectic temperature; reducing a temperature of the material to below the material eutectic temperature; decreasing the chamber pressure; adding heat to the material, while maintaining the material temperature below the material eutectic temperature for a period of time; after the period of time, heating the material above its eutectic temperature; and sterilizing the vacuum chamber. The vacuum chamber is sterilized by vaporizing liquid sterilant located in a liquid sterilant supply, wherein the liquid sterilant supply has a higher pressure than the vacuum chamber; and sterilizing the vacuum chamber with the vaporized sterilant.

The above described and other features are exemplified by the following Figures and detailed description.

### BRIEF DESCRIPTION OF THE DRAWINGS

Refer now to the drawings, which are exemplary, not limiting, and wherein like elements are numbered alike.

FIG. 1 is a schematic view of an embodiment of a vacuum system

FIG. 2 is a schematic view of an embodiment of vacuum system including a bulk liquid sterilant supply.

### DETAILED DESCRIPTION

Freeze-drying, also termed “lyophilization”, typically comprises three stages; the “freezing stage”, the “primary drying stage”, and the “secondary drying” stage. In a typical freeze-drying process, an aqueous solution or product containing, for example, a drug and various formulation aids, or “excipients”, is filled into glass vials or bulk trays, and the vials or trays are loaded onto temperature-controlled shelves within the drying chamber, the terminal operation of which is the placement of a stopper in the vials (not applicable if bulk drying) to seal the contents therein and releasing to atmospheric pressure for removal.

The materials involved in lyophilization processes are usually biological in nature (bacteria, fungi, viruses, spores or tissue). It is not unusual that these biological materials can pose health risks to humans, and therefore most lyophilization processes involving biologics include at least one process step called sterilization, in which any biologic materials inside the lyophilization chambers are killed. This process is carried out between cycle to prevent cross contamination between batches and also reduce the bioburden within the product.

The predominant sterilization technique involves the use of high temperature, high pressure steam. Typically this requires a supply of steam at about 125° C. and pressures of about 30 psi, which is introduced into the lyophilizer chambers and all other internally wetted surfaces for some period of time after the lyophilized vials (and/or trays) have been removed. The soak time is determined by experiment, depending on the biologic involved, as validated to insure a sufficient percentage of any active biologics have been killed (e.g., killed down to the desired concentration).

In general terms, the present method utilizes the existing vacuum in the lyophilizer to evaporate a prescribed dose of liquid sterilant (e.g., a container of which can be passively connected to the lyophilizer, such as via a length of tubing (e.g., capillary tubing)). The driving mechanism for the evaporation is the pressure differential between the source container (e.g., at atmospheric pressure) and the lyophilizer chambers (e.g., at 2 to 50 millitorr (mT) or so). Once the stoppered vials have been removed from the chamber and the chamber door is then closed and the chamber evacuated, the terminal sterilization can be performed such that the liquid sterilant is vaporized and introduced into the vacuum chamber. It may also be possible to perform this sterilization method while the stopped vials (e.g., any closed containers) are still within the lyophilizer as the temperatures utilized with this process should not cause product degradation. This is assuming the vials are fully closed (e.g., stoppered) and positively sealed. Sufficient concentration of the sterilant and residence time is controlled to enable the desired level of sterilization (e.g., to a kill level required by regulation for the particular material).

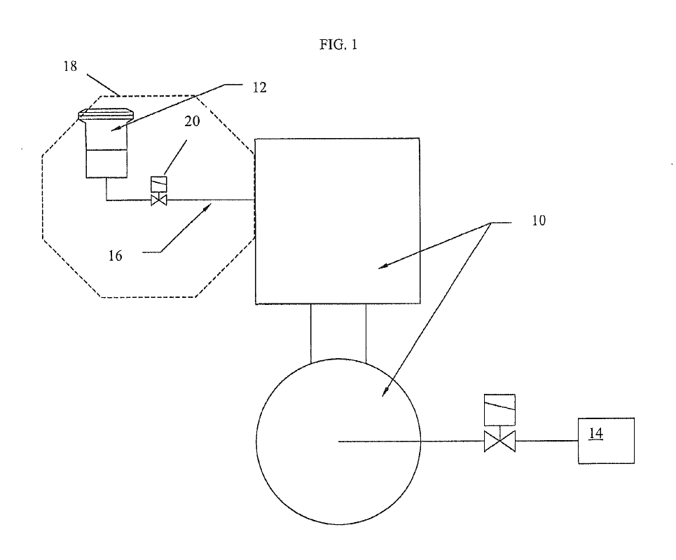

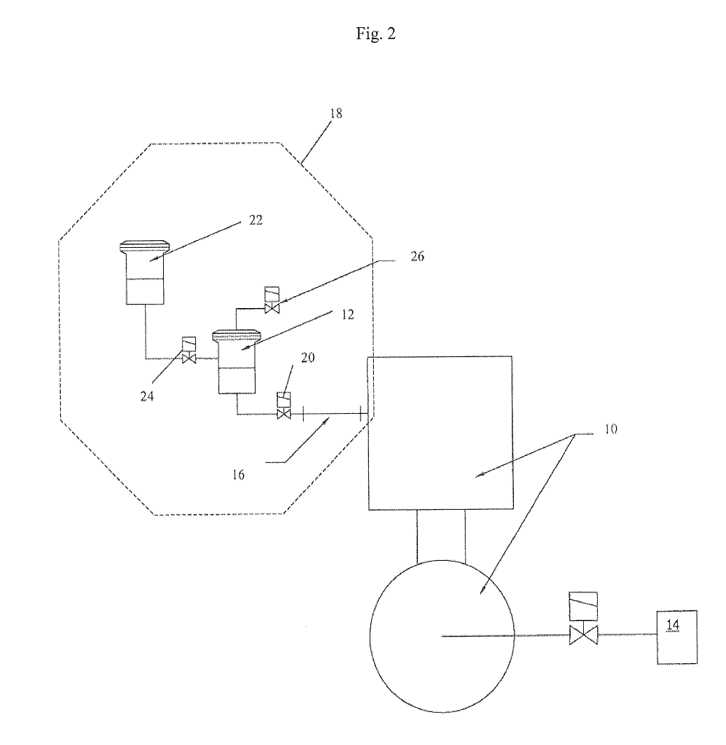

Referring to FIGS. 1 and 2, the system comprises a vacuum chamber(s) (e.g., a lyophilizer with two chambers connected via a vapor port) (10), a vacuum pump (14) in operable communication with the vacuum chamber (10), and a sterilant delivery system (18) in operable communication with the vacuum chamber (10). The sterilant delivery system (18) comprised a sterilant supply (12), a valve (20), and a metering device (16). As can be seen from FIG. 2, the sterilant delivery system (18) can further comprise a bulk sterilant supply (22) connected to the dose sterilant supply (12) via the optional valve (24). The dose sterilant supply (12) can be in fluid communication with a dose reservoir vent (26).

Depending upon the sterilant supply (12), the metering device (16) can comprise various dosage control element(s). Some exemplary metering devices comprise reservoir(s) (e.g., overflow container, volume controlled container, and so forth), tube(s) (such as capillary tubes), valves (e.g., metering valves, expansion valves, and so forth), orifices, and the like, as well as combinations comprising at least one of the foregoing metering devices. For example, if the sterilant supply (12) only contains the desired dosage of the sterilant, the metering device can be a capillary tube in operable communication with the sterilant supply (12) such that when a vacuum is pulled from the chamber (10), a pressure differential is created across the capillary tube, causing the flow of the sterilant through the valve (20) and also (e.g., simultaneously) causing the sterilant to vaporize within the capillary tube and then to be delivered into the chamber (10). In another embodiment, a reservoir can be located before the sterilant supply (12) and the metering device (20) such that sterilant can be allowed to flow from the reservoir into the sterilant supply until the desired dosage is reached. Then a valve between the sterilant supply (12) and the reservoir is closed and a vent (e.g., vent (26)) on the reservoir (if present) is also closed. Upon application of the vacuum, the valve between the sterilant supply and the metering device is opened and the sterilant is vaporized as it is drawn into the chamber.

The metering device (20) can be any device that will meter the flow and allow the particular sterilant to vaporize under the vacuum conditions that will be employed in the vacuum chamber (10) without freezing the sterilant in the sterilant supply (12) or the metering device (20). In other words, the metering device is sized such that liquid sterilant can be introduced into the vacuum chamber without solidifying remaining liquid sterilant. This allows the sterilant to vaporize within the metering device or upon entry of the vacuum chamber.

In some embodiments, the sterilant supply is maintained at atmospheric pressure, while the vacuum chamber is at a reduced pressure compared to the sterilant supply. Once the valve is opened to pass the sterilant out of the sterilant supply, the sterilant is exposed to a reduced pressure that is less than the vapor pressure of the sterilant. Furthermore, the pressure is maintained below the vapor pressure of the sterilant. For example, the vacuum chamber can be maintained at a pressure of less than or equal to 500 mT, specifically, less than or equal to 200 mT, more specifically, less than or equal to 100 mT, and even less than or equal to 60 mT such as 2 mT to 50 mT.

The sterilant can be any liquid (e.g., vaporizable liquid) that will attain the desired sterilization affects (e.g., is toxic to the material(s) being controlled). The particular sterilant utilized in the system is dependent upon the contaminant(s) to be sterilized. Other considerations may be the vapor pressure of the sterilant, the amount of heat absorbed when vaporizing, and/or its affect on the system equipment. Desirably, the sterilant is not a material that will degrade the vacuum chamber and associated components and equipment. Exemplary sterilants include various disinfectants such as hydrogen peroxide, ethylene oxide, peracetic acid, chlorine dioxide, and the like, as well as combinations comprising at least one of the foregoing sterilants. For example, a sterilant can comprise 1 weight percent (wt %) to 50 wt % hydrogen peroxide, specifically, 5 wt % to 40 wt %, more specifically, 15 wt % to 30 wt %, and yet more specifically 20 wt % to 25 wt %, wherein the weight percent is based upon a total weight of the sterilant. In some embodiments, the balance is a carrier (e.g., water), and/or another sterilant. Alternatively, or in addition, the sterilant can comprise up to 10 wt % peracetic acid, specifically, 0.01 wt % to 10 wt %, more specifically 2 wt % to 7 wt %, wherein the weight percent is based upon a total weight of the sterilant.

The method, therefore, can comprise vaporizing the liquid sterilant at a rate that avoids freezing of the sterilant, introducing the vapor to the vacuum chamber, and retaining the vapor in the vacuum chamber for a sufficient amount of time to attain the desired sterilization results (e.g., kill rate and amount). With reference to FIG. 1, a liquid sterilant can be located in a sterilant supply (12). A vacuum is pulled on the vacuum chamber (10) by the vacuum pump (14). Sterilant is then introduced to the metering device (16), e.g., a tube having a sufficient diameter and length such that, at the pressure differential between the sterilant supply (12) and the vacuum chamber (10) (and hence also the tube), the sterilant vaporizes at a rate that retains the temperature of the sterilant above its freezing temperature. In other words, the sterilant vaporization rate is controlled to prevent freezing of the liquid sterilant. The pressure differential between the sterilant supply (12) and the vacuum chamber (10) is sufficient to drive the sterilant from the sterilant supply (12) into the vacuum chamber (10). After the vacuum chamber (10) and the sterilant supply (12) have reached steady state (i.e., the pressure in the vacuum chamber (10) and the sterilant supply (12) have equalized), system can be released (e.g., pressure brought to atmospheric pressure and the vacuum chamber opened), or the system can be maintained closed (e.g., not opened to the atmosphere) for a sufficient residence time of the sterilant to attain the desired sterilization effects. Optionally, container(s) can be simultaneously sterilized by retaining them in the vacuum chamber during the sterilization.

Since the present process employs the pressure differential between the vacuum chamber and the sterilant supply to attain vaporization, over-pressurization and high temperatures are avoided. Although heat can optionally be added to the system, this process can be affected without the addition of heat, by relying solely on the pressure differential and the fluid dynamic controls imparted by the metering device.

In one embodiment, the system can be a lyophilization system. The process can comprise loading the material (e.g., the product to be freeze dried) into a vacuum chamber and reducing the temperature of the material to below its eutectic temperature. The vacuum chamber pressure can then be reduced, e.g., to less than or equal to 200 mT, specifically, less than or equal to 100 mT. Once the vacuum chamber is at the desired pressure, primary drying can proceed by adding heat to the material (e.g., shelves of the chamber can be heated), while maintaining the material temperature below its eutectic temperature. Once sufficient moisture is removed to prevent cake collapse, the secondary drying step can be performed to remove water bound within the product by heating the material above its eutectic temperature, but desirably to below a temperature that will degrade the product activity, until the material is sufficiently dried (e.g., to a residual moisture of less than or equal to 5 wt %, specifically less than or equal to 2 wt %, based upon the total weight of the material).

If the product is in containers, the containers can be closed (e.g., vials stoppered, lids closed, etc.). Then, the product can be removed from the chamber. If sterilization of the outside of the container(s) is also desired, the containers can remain in the chamber. The temperature of the chamber can remain at the raised temperature, or the temperature can be reduced to room temperature (e.g., for efficiency reasons). The liquid sterilant can then be vaporized and introduced to the chamber as described above. If sterilization of the product container(s) is not desired, or the product is not located in a closable container, the product can be removed from the chamber before the sterilization process.

The following examples are exemplary, merely intended to illustrate some embodiments of the disclosed system and method.

### EXAMPLES

### Example 1

Sterilization of a 166 liter (L) system comprising G. Stearothermophilus Biological indicators with a 10⁶ population was tested using 4.5 milliliters (ml) of a sterilant solution comprising hydrogen peroxide and peracetic acid (22 wt % and 4.5 wt %, respectively) in a liquid state. The process included an initial evacuation of the vacuum chamber down to 50 millitorr (mT). The sterilant was then vaporized by opening a dose valve such that the sterilant could pass from the sterilant supply (a closed vessel that was at atmospheric pressure) into a capillary tube (having a size of 0.02 inch diameter and 60 inches in length) that was at the same pressure as the vacuum chamber (50 mT). A dose time of 5 minutes was allowed to pass. The vacuum pump and vacuum lines were activated for 30 seconds (sec), and then the system was allowed to set and “sterilize” for 60 minutes. The system was then evacuated to 100 mT and released to 700 Torr with room air (although another suitable gas such as nitrogen could be used) 5 times to remove any residual vapor. Upon completing the cycle, the biological indicators as well as a standard were incubated for 24 hours. All “sterilized” indicators were killed and the standard indicated successful incubation, thus proving successful sterilization of the system, the viability of the process and the accuracy of the dose.

### Example 2

After successful delivery of a dose of 4.5 milliliters (ml) of a sterilant solution into a system with a volume of approximately 166 liters (L), the same theory was tested on a larger system having a volume of 3,500 L, i.e., approximately 30 times the volume of the smaller test system, using the same capillary tube of Example 1. Therefore the charge volume of sterilant was adjusted to 135 ml. The same procedure as used for Example 1 was also used here, except the dose time was extended to 10 minutes to allow the larger volume dose to be administered. Again, all “sterilized” indicators were killed and the standard indicated successful incubation, thus proving the larger system and dose.

Biological indicators were placed in eight different locations that varied in perceived difficulty to penetrate. The cycle was run manually with the same process as in Example 1 including evacuation pressures and exposure times.

A major concern of a larger system was whether the sterilant would freeze within the feed device (sterilant supply, or metering device (e.g., reservoir)) before entering the system. To initially help prevent this, the feed capillary was passed through a room temperature water bath in attempts to warm the passing sterilant as it expands. The testing showed that the area immediately exiting the supply capillary cooled to the point of actually frosting up the body of the first sanitary valve. Based on the coverage results, the sterilant still vaporized and provided the necessary kill factors. The heat provided by the capillary bath was not sufficient most likely due to the flow rate of the sterilant and more localized expansion at the end of the capillary.

Both objectives of successful sterilization and adequate dose supply were achieved. This was in addition to confirmation that the cooling effect of the liquid sterilant in a larger dose, although it is present, does not affect the process. The metering device can be adjusted to control the flow rate, and hence the vaporization rate, and therefore the cooling effect. Freezing of the sterilant can be avoided.

It is clear from the above, that this process can be automated such that when sterilization of a vacuum system is desired, a controller can open the necessary valves to allow a metered amount of sterilant to be vaporized and introduced to the chamber.

The present system and process addresses many issues associated with other sterilization processes, such as: (i) the requirement that the vacuum chamber (e.g., lyophilizer) being capable of withstanding temperature and pressure extremes (as well as attendant machine costs and leadtimes); (ii) extended cycle times; (iii) facilities infrastructure required to generate an ample supply of clean steam; (iv) problems and difficulties with using ethylene oxide; (v) need for an expensive hydrogen peroxide generator; and (vi) problems with over pressurizing the vacuum system (e.g., lyophilizer system) which is designed and rated for vacuum conditions.

The present system and method utilizes the existing vacuum capabilities of the vacuum system (e.g., lyophilizer system) to disperse the sterilant therein. Hence, temperature and pressure extremes, over pressurizing requirements, and facilities infrastructure for steam supply, are all eliminated. Although ethylene oxide can be utilized in this process, other sterilants are also possible. The present dispensing method does not require the addition of a pressurized source to achieve vaporization by atomization. As a result, it is believed that higher concentrations of sterilant in the system can be achieved as compared to systems using increased pressure (above atmospheric). Also, there is no risk of pressurizing the lyophilizer, as the evaporation of the sterilant cannot drive pressures within the lyophilizer above atmospheric pressure.

In one embodiment, a vacuum system comprises: a vacuum chamber; a vacuum source in operable communication with the vacuum chamber; a liquid sterilant supply; and a metering device. The sterilant supply is in operable communication with the vacuum chamber via the metering device. The metering device has a size such that liquid sterilant can be introduced into the vacuum chamber as sterilant vapor.

In one embodiment, a method of lyophilization comprises: loading into a vacuum chamber having a chamber pressure, a material having a material eutectic temperature; reducing a temperature of the material to below the material eutectic temperature; decreasing the chamber pressure; adding heat to the material, while maintaining the material temperature below the material eutectic temperature for a period of time; after the period of time, heating the material above its eutectic temperature; and sterilizing the vacuum chamber. The vacuum chamber is sterilized by vaporizing liquid sterilant located in a liquid sterilant supply, wherein the liquid sterilant supply has a higher pressure than the vacuum chamber; and sterilizing the vacuum chamber with the vaporized sterilant.

In one embodiment, a method of sterilizing a vacuum system, comprises: creating a vacuum in a vacuum chamber such that a pressure differential is formed between the vacuum chamber and a liquid sterilant supply, and wherein the liquid sterilant supply has a higher pressure than the vacuum chamber; vaporizing liquid sterilant; and sterilizing the vacuum chamber with the vaporized sterilant.

In the various embodiments, (i) the system is a lyophilizer system; and/or (ii) the metering device is selected from the group consisting of a tube, a valve, an orifice, as well as combinations comprising at least one of the foregoing; and/or (iii) the sterilant supply comprises sterilant selected from the group consisting of hydrogen peroxide, ethylene oxide, peracetic acid, and the like, as well as combinations comprising at least one of the foregoing sterilants; and/or (iv) the sterilant comprises 1 wt % to 50 wt % hydrogen peroxide, wherein the weight percent is based upon a total weight of the sterilant; and/or (v) the sterilant comprises 5 wt % to 40 wt % hydrogen peroxide; and/or (vi) the sterilant comprises 15 wt % to 30 wt % hydrogen peroxide; and/or (vii) the sterilant further comprises peracetic acid; and/or (viii) the sterilant comprises 0.01 wt % to 10 wt % peracetic acid; and/or (ix) the peracetic acid is present in an amount of 2 wt % to 7 wt %; and/or (x) the metering device is designed to allow vaporization of the liquid sterilant such that a temperature of the liquid sterilant remains above a freezing temperature of the liquid sterilant; and/or (xi) the liquid sterilant remains at a temperature above a freezing temperature of the liquid sterilant; and/or (xii) the method further comprises passing the liquid sterilant from the liquid sterilant supply into a tube having a sufficient size to allow vaporization of the liquid sterilant without allowing the temperature to decrease to below the freezing temperature; and/or (xiii) the method further comprises reducing the pressure of the vacuum chamber once a complete dose of liquid sterilant has exited the liquid sterilant supply; and/or (xiv) decreasing the chamber pressure comprises decreasing the chamber pressure to less than or equal to 200 mT; and/or (xv) the material is maintained at below the material eutectic temperature until a residual moisture of less than or equal to 5 wt % in the material is attained, wherein the weight percent is based upon a total weight of the material; and/or (xvi) the residual moisture is less than or equal to 2 wt %; and/or (xvii) the liquid sterilant remains at a temperature above a freezing temperature of the liquid sterilant.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

What is claimed is:

1. A method of sterilizing a vacuum system, comprising:
creating a vacuum in a vacuum chamber such that a pressure differential is formed between the vacuum chamber and a liquid sterilant supply, and wherein the liquid sterilant supply has a higher pressure than the vacuum chamber;
vaporizing liquid sterilant before it enters the vacuum chamber; and
sterilizing the vacuum chamber with the vaporized sterilant.

2. The method of claim 1, wherein the liquid sterilant remains at a temperature above a freezing temperature of the liquid sterilant.

3. The method of claim 1, further comprising passing the liquid sterilant from the liquid sterilant supply into a tube having a sufficient size to allow vaporization of the liquid sterilant without allowing the temperature to decrease to below the freezing temperature.

4. The method of claim 1, further comprising reducing the pressure of the vacuum chamber once a complete dose of liquid sterilant has exited the liquid sterilant supply.

5. The method of claim 1, wherein the vaporizing is attained solely via the pressure differential and fluid dynamic controls.

6. The method of claim 1, comprising vaporizing the liquid sterilant without the addition of heat.

7. A method of lyophilization, comprising:
loading into a vacuum chamber having a chamber pressure, a material having a material eutectic temperature;
reducing a temperature of the material to below the material eutectic temperature;
decreasing the chamber pressure;
adding heat to the material, while maintaining the material temperature below the material eutectic temperature for a period of time;
after the period of time, heating the material above its eutectic temperature;
sterilizing the vacuum chamber by
vaporizing liquid sterilant and introducing the vaporized liquid sterilant to the vacuum chamber, wherein a liquid sterilant supply has a higher pressure than the vacuum chamber; and
sterilizing the vacuum chamber with the vaporized sterilant.

8. The method of claim 7, wherein decreasing the chamber pressure comprises decreasing the chamber pressure to less than or equal to 200 mT.

9. The method of claim 7, wherein the material is maintained at below the material eutectic temperature until a residual moisture of less than or equal to 5 wt % in the material is attained, wherein the weight percent is based upon a total weight of the material.

10. The method of claim 7, wherein the residual moisture is less than or equal to 2 wt %.

11. The method of claim 7, wherein the liquid sterilant remains at a temperature above a freezing temperature of the liquid sterilant.

12. The method of claim 7, comprising vaporizing the liquid sterilant via the pressure differential and fluid dynamic controls.

13. The method of claim 7, comprising vaporizing the liquid sterilant without the addition of heat.

14. A method of sterilizing a vacuum system, comprising:
creating a vacuum in a vacuum chamber such that a pressure differential is formed between the vacuum chamber and a liquid sterilant supply, and wherein the liquid sterilant supply has a higher pressure than the vacuum chamber;
vaporizing liquid sterilant as it passes through a tube having a sufficient size to allow vaporization of the liquid sterilant without allowing the temperature to decrease to below the freezing temperature;
introducing the vaporized liquid sterilant to the vacuum chamber; and
sterilizing the vacuum chamber with the vaporized sterilant.

15. The method of claim 14, wherein the tube is a capillary tube.

16. The method of claim 14, comprising vaporizing the liquid sterilant via the pressure differential and fluid dynamic controls.

17. The method of claim 14, comprising vaporizing the liquid sterilant without the addition of heat.
